(19) United States
(12) Patent Application Publication
(10) Pub. No.: US 2024/0369541 A1
(43) Pub. Date: Nov. 7, 2024

(54) Artificial Intelligence Enhanced Real Time Biological Optimization and Health Monitoring for Space Travel

(71) Applicant: CREATIVE MEDICAL TECHNOLOGIES, INC., Phoenix, AZ (US)

(72) Inventors: Thomas Ichim, San Diego, CA (US); Courtney Bartlett, Niceville, FL (US); Timothy Warbington, Phoenix, AZ (US); Amit Patel, Salt Lake City, UT (US)

(73) Assignee: CREATIVE MEDICAL TECHNOLOGIES, INC., Phoenix, AZ (US)

(21) Appl. No.: 18/647,395

(22) Filed: Apr. 26, 2024

Related U.S. Application Data
(60) Provisional application No. 63/463,993, filed on May 4, 2023.

(51) Int. Cl. G01N 33/50; B64G 1/52

(52) U.S. Cl. CPC G01N 33/5091; B64G 1/525

(57) ABSTRACT

Disclosed are methods and means of optimizing health of a subject during space travel comprising real time monitoring and intervention guided by automated and/or semiautomated means driven by a machine learning artificial intelligence-based system. In one embodiment the invention discloses the integration of genetic factors, biological properties, microbiome diversity, regenerative activity, and real time physiological changes in order to provide optimum interventions which result in increased health upon return from the space mission, as well as provide ability for said astronaut to endure space travel of increased durations.

### CROSS-REFERENCE TO RELATED APPLICATIONS

The present application claims benefit of Provisional Patent Application Ser. No. 63/463,993, filed on May 4, 2023, entitled ARTIFICIAL INTELLIGENCE ENHANCED REAL TIME BIOLOGICAL OPTIMIZATION AND HEALTH MONITORING FOR SPACE TRAVEL, the contents of which are incorporated herein by reference in its entirety.

### FIELD OF THE INVENTION

The teachings herein relates to the use of artificial intelligence for enhanced optimization and monitoring of space travelers.

### BACKGROUND OF THE INVENTION

It is known that space travel exposes the body to numerous known and unknown risks that are not encountered in terrestrial life. For example, reaching escape velocity to exit the earth's gravitational field requires the body be exposed to conditions of hyper gravity, which is unavoidable. Subsequently, numerous physiological and biologic parameters are altered in during the minimum or zero gravity environments of space. Additionally, space flights leaving the outer limits of the ionosphere subject the astronaut to various cosmic radiations whose effects are dissimilar to classical terrestrially measured radiation such as alpha, beta and gamma rays.

Artificial intelligence representations, and more specifically, application of said representations have been used for understanding multiple complex systems and providing real-time solutions and responses. To date the use of artificial intelligence has not been applied to space travel associated medical conditions and responding to said medical conditions or alterations in homeostasis that can alter the health of the astronaut. Automated planning and scheduling is a branch of artificial intelligence (AI) that concerns the realization of strategies or action sequences, typically for execution by intelligent agents, autonomous robots, and unmanned vehicles. Unlike classical control and classification problems, solutions are complex and must be discovered and optimized in multidimensional space. Planning is also related to decision theory. A planning problem generally comprises the following main elements: a finite set of facts, the initial state (a set of facts that are true initially), a finite set of action operators (with precondition and effects), and a goal condition. An action operator maps a state into another state. In the classical planning, the objective is to find a sequence of action operators (or planning action) that, when applied to the initial state, will produce a state that satisfies the goal condition.

### SUMMARY OF THE INVENTION

Preferred methods include embodiments for maintaining optimum health of an astronaut during a space mission comprising the steps of: a) establishing a genetic and biological background on said astronaut; b) establishing a baseline level of physiological and biological features of said astronaut; c) monitoring said physiological and biological features in real time during space flight; d) intervening based on algorithms and/or artificial intelligence mediated instructions; and e) adjusting the interventions based on physiological and/or biological responses to said intervention.

Preferred methods include embodiments wherein said genetic background comprises gene polymorphisms associated with augmented or diminished expression of a gene.

Preferred methods include embodiments wherein said genetic background comprises gene polymorphisms associated with blockade of expression of a gene.

Preferred methods include embodiments wherein said genetic background is a polymorphism altering function of one or more organs.

Preferred methods include embodiments wherein said genetic background is a polymorphism which modulates activity of a transcription factor.

Preferred methods include embodiments wherein transcription factor is a nuclear receptor.

Preferred methods include embodiments wherein said genetic background comprises a reduces generation of cells with regenerative potential.

Preferred methods include embodiments wherein said cells with regenerative potential are endogenous stem cells.

Preferred methods include embodiments wherein said endogenous stem cells are capable of asymmetric division.

Preferred methods include embodiments wherein said asymmetric division is associated with activation of the telomerase enzyme.

Preferred methods include embodiments wherein said asymmetric division occurs in a manner in which said endogenous stem cell makes a copy of itself, as well as a cell with differentiated characteristics.

Preferred methods include embodiments wherein said differentiated characteristics are protein surface markers.

Preferred methods include embodiments wherein said differentiated characteristics are lineage specific proteins.

Preferred methods include embodiments wherein said asymmetric division can occur for at least 10 cycles.

Preferred methods include embodiments wherein said endogenous stem cells are found in areas of relative hypoxia.

Preferred methods include embodiments wherein said endogenous stem cells are primarily found in the G0 state of cell cycle.

Preferred methods include embodiments wherein said endogenous stem cells enter cell cycle in response to injury.

Preferred methods include embodiments wherein said injury is associated with necrotic cell death.

Preferred methods include embodiments wherein said injury is associated with pyroptotic cell death.

Preferred methods include embodiments wherein said injury is associated with ferroptotic cell death.

Preferred methods include embodiments wherein said endogenous stem cell possesses higher levels of antioxidant enzymes as compared to non-stem cell cells.

Preferred methods include embodiments wherein said antioxidant enzyme is manganese dependent superoxide dismutase.

Preferred methods include embodiments wherein said antioxidant enzyme is zinc dependent superoxide dismutase.

Preferred methods include embodiments wherein said antioxidant enzyme is xanthine oxidase.

Preferred methods include embodiments wherein said antioxidant enzyme is catalase.

Preferred methods include embodiments wherein said endogenous stem cell possesses higher levels of immune suppressive molecules as compared to non-stem cell cells.

Preferred methods include embodiments wherein said immune suppressive molecule is indolamine 2,3 dioxygenase.

Preferred methods include embodiments wherein said immune suppressive molecule is Fas ligand.

Preferred methods include embodiments wherein said immune suppressive molecule is indolamine 2,3 dioxygenase.

Preferred methods include embodiments wherein said immune suppressive molecule is CTLA-4.

Preferred methods include embodiments wherein said immune suppressive molecule is BTLA-4.

Preferred methods include embodiments wherein said immune suppressive molecule is inducible nitric oxide synthetase.

Preferred methods include embodiments wherein said immune suppressive molecule is prostaglandin E2.

Preferred methods include embodiments wherein said immune suppressive molecule is HLA-G.

Preferred methods include embodiments wherein said immune suppressive molecule is soluble HLA-G.

Preferred methods include embodiments wherein said immune suppressive molecule is TREM-1.

Preferred methods include embodiments wherein said immune suppressive molecule is soluble fas ligand.

Preferred methods include embodiments wherein said immune suppressive molecule is TNF-alpha.

Preferred methods include embodiments wherein said immune suppressive molecule is TRAIL.

Preferred methods include embodiments wherein said immune suppressive molecule is lymphotoxin.

Preferred methods include embodiments wherein said immune suppressive molecule is PDGF-BB.

Preferred methods include embodiments wherein said immune suppressive molecule is thymosin beta 4.

Preferred methods include embodiments wherein said immune suppressive molecule is syndecan.

Preferred methods include embodiments wherein said immune suppressive molecule is interleukin 1 receptor antagonist.

Preferred methods include embodiments wherein said endogenous stem cells suppress proliferation of immune cells.

Preferred methods include embodiments wherein said immune cells are inflammatory.

Preferred methods include embodiments wherein said inflammatory immune cells are Th1 cells.

Preferred methods include embodiments wherein said inflammatory immune cells are Th17 cells.

Preferred methods include embodiments wherein said inflammatory immune cells are Th9 cells.

Preferred methods include embodiments wherein said inflammatory immune cells are gamma delta T cells.

Preferred methods include embodiments wherein said inflammatory immune cells are NKT cells.

Preferred methods include embodiments wherein said inflammatory immune cells are type 1 macrophages.

Preferred methods include embodiments wherein said inflammatory immune cells are type 1 astrocytes.

Preferred methods include embodiments wherein said inflammatory immune cells are type 1 neutrophils.

Preferred methods include embodiments wherein said endogenous stem cells express MCP-1.

Preferred methods include embodiments wherein said endogenous stem cells express G-CSF.

Preferred methods include embodiments wherein said endogenous stem cells express MIG-1.

Preferred methods include embodiments wherein said endogenous stem cells express MIP-1 alpha.

Preferred methods include embodiments wherein said endogenous stem cells express MIP-1 beta.

Preferred methods include embodiments wherein said endogenous stem cells express RANTES.

Preferred methods include embodiments wherein said endogenous stem cells express TIMP-1.

Preferred methods include embodiments wherein said endogenous stem cells express TIMP-2.

Preferred methods include embodiments wherein said endogenous stem cells express TNF-alpha.

Preferred methods include embodiments wherein said endogenous stem cells express TNF-beta.

Preferred methods include embodiments wherein said endogenous stem cells express BDNF.

Preferred methods include embodiments wherein said endogenous stem cells express acidic FGF.

Preferred methods include embodiments wherein said endogenous stem cells express basic FGF.

Preferred methods include embodiments wherein said endogenous stem cells express BMP-4.

Preferred methods include embodiments wherein said endogenous stem cells express BMP-5.

Preferred methods include embodiments wherein said endogenous stem cells express BMP-7.

Preferred methods include embodiments wherein said endogenous stem cells express Nerve Growth Factor.

Preferred methods include embodiments wherein said endogenous stem cells express FGF-4.

Preferred methods include embodiments wherein said endogenous stem cells express FGF-7.

Preferred methods include embodiments wherein said endogenous stem cells express GDF-11.

Preferred methods include embodiments wherein said endogenous stem cells express GDF-15.

Preferred methods include embodiments wherein said endogenous stem cells express GDNF.

Preferred methods include embodiments wherein said endogenous stem cells express Growth Hormone.

Preferred methods include embodiments wherein said endogenous stem cells express HB-EGF.

Preferred methods include embodiments wherein said endogenous stem cells express IGFBP-1.

Preferred methods include embodiments wherein said endogenous stem cells express IGFBP-2.

Preferred methods include embodiments wherein said endogenous stem cells express IGFBP-3

Preferred methods include embodiments wherein said endogenous stem cells express IGFBP-4.

Preferred methods include embodiments wherein said endogenous stem cells express IGBP-6.

Preferred methods include embodiments wherein said endogenous stem cells express IGF-1.

Preferred methods include embodiments wherein said endogenous stem cells express NT-3.

Preferred methods include embodiments wherein said endogenous stem cells express NT-4.

Preferred methods include embodiments wherein said endogenous stem cells express Osteoprotegerin.

Preferred methods include embodiments wherein said endogenous stem cells express PDGF-AA.

Preferred methods include embodiments wherein said endogenous stem cells express PIGF.

Preferred methods include embodiments wherein said endogenous stem cells express stem cell factor.

Preferred methods include embodiments wherein said endogenous stem cells express stem cell factor receptor.

Preferred methods include embodiments wherein said endogenous stem cells express TGF-alpha.

Preferred methods include embodiments wherein said endogenous stem cells express TGF-beta.

Preferred methods include embodiments wherein said endogenous stem cells express TGF-beta 3.

Preferred methods include embodiments wherein said endogenous stem cells express VEGF.

Preferred methods include embodiments wherein said endogenous stem cells express VEGF-R2.

Preferred methods include embodiments wherein said endogenous stem cells express VEGF-R3.

Preferred methods include embodiments wherein said endogenous stem cells express VEGF-D.

Preferred methods include embodiments wherein said endogenous stem cells express 6Ckine.

Preferred methods include embodiments wherein said endogenous stem cells express axl.

Preferred methods include embodiments wherein said endogenous stem cells express BTC.

Preferred methods include embodiments wherein said endogenous stem cells express CCL28.

Preferred methods include embodiments wherein said endogenous stem cells express CXCL16.

Preferred methods include embodiments wherein said endogenous stem cells express ENA-78.

Preferred methods include embodiments wherein said endogenous stem cells express Eotaxin-3.

Preferred methods include embodiments wherein said endogenous stem cells express GCP-2.

Preferred methods include embodiments wherein said endogenous stem cells express GRO.

Preferred methods include embodiments wherein said endogenous stem cells express HCC-1.

Preferred methods include embodiments wherein said endogenous stem cells express HCC-4.

Preferred methods include embodiments wherein said endogenous stem cells express interleukin-9.

Preferred methods include embodiments wherein said endogenous stem cells express interleukin-17F.

Preferred methods include embodiments wherein said endogenous stem cells express interleukin-18.

Preferred methods include embodiments wherein said endogenous stem cells express interleukin-28A.

Preferred methods include embodiments wherein said endogenous stem cells express interleukin-29.

Preferred methods include embodiments wherein said endogenous stem cells express interleukin-31.

Preferred methods include embodiments wherein said endogenous stem cells express IP-10.

Preferred methods include embodiments wherein said endogenous stem cells express I-TAC.

Preferred methods include embodiments wherein said endogenous stem cells express osteopontin.

Preferred methods include embodiments wherein said endogenous stem cells express PARC.

Preferred methods include embodiments wherein said endogenous stem cells express PF4.

Preferred methods include embodiments wherein said endogenous stem cells express TSLP.

Preferred methods include embodiments wherein said endogenous stem cells express ALCAM.

Preferred methods include embodiments wherein said endogenous stem cells express CEACAM-1.

Preferred methods include embodiments wherein said endogenous stem cells express Angiogenin.

Preferred methods include embodiments wherein said endogenous stem cells express ErbB3.

Preferred methods include embodiments wherein said endogenous stem cells express HVEM.

Preferred methods include embodiments wherein said endogenous stem cells express TRANCE.

Preferred methods include embodiments wherein patients possessing genetic or biological predisposition to lower numbers of endogenous stem cells are assessed for levels of endogenous stem cells through quantification of circulating stem cells.

Preferred methods include embodiments wherein said circulating endogenous stem cells are assessed for by ability of mononuclear cells extracted from blood to form hematopoietic colonies when cultured on a semisolid media containing hematopoietic growth factors.

Preferred methods include embodiments wherein said semisolid media is methylcellulose.

Preferred methods include embodiments wherein said hematopoietic growth factors are selected from a group containing: a) G-CSF; b) GM-CSF; c) interleukin-3; d) steel factor; e) interleukin-11; f) thrombopoietin; g) stem cell factor; and h) flt3-ligand.

Preferred methods include embodiments wherein said hematopoietic growth factor is hepatocyte growth factor.

Preferred methods include embodiments wherein said hematopoietic growth factor is VEGF.

Preferred methods include embodiments wherein said hematopoietic growth factor is acidic FGF.

Preferred methods include embodiments wherein said hematopoietic growth factor is basic FGF.

Preferred methods include embodiments wherein said hematopoietic growth factor is stromal derived factor=1.

Preferred methods include embodiments wherein said hematopoietic growth factor is endoglin.

Preferred methods include embodiments wherein said hematopoietic growth factor is angiopoietin.

Preferred methods include embodiments wherein said hematopoietic growth factor is soluble jagged.

Preferred methods include embodiments wherein said hematopoietic growth factor is monocyte colony stimulating factor.

Preferred methods include embodiments wherein said hematopoietic colonies consist of monocytes.

Preferred methods include embodiments wherein said hematopoietic colonies consist of erythroid progenitors.

Preferred methods include embodiments wherein said hematopoietic colonies consist of erythrocytes.

Preferred methods include embodiments wherein said hematopoietic colonies consist of neutrophils.

Preferred methods include embodiments wherein said hematopoietic colonies consist of eosinophils.

Preferred methods include embodiments wherein said hematopoietic colonies consist of basophils.

Preferred methods include embodiments wherein said hematopoietic colonies consist of thrombocytes.

Preferred methods include embodiments wherein said hematopoietic colonies consist of megakaryocytes.

Preferred methods include embodiments wherein said hematopoietic colonies consist of lymphoid progenitors.

Preferred methods include embodiments wherein said hematopoietic colonies consist of T cells.

Preferred methods include embodiments wherein said hematopoietic colonies consist of B cells.

Preferred methods include embodiments wherein said hematopoietic colonies consist of natural killer cells.

Preferred methods include embodiments wherein said hematopoietic colonies consist of natural killer T cells.

Preferred methods include embodiments wherein said hematopoietic colonies consist of gamma delta T cells.

Preferred methods include embodiments wherein based on abnormalities of hematopoietic stem cell number subjects are treated with various compounds to ensure proper function and number of said hematopoietic stem cells.

Preferred methods include embodiments wherein said number of hematopoietic stem cells is quantified by colony forming units.

Preferred methods include embodiments wherein said number of hematopoietic stem cells is quantified by number of CD34 positive cells in circulation.

Preferred methods include embodiments wherein said number of hematopoietic stem cells is quantified by number of CD133 positive cells in circulation.

Preferred methods include embodiments wherein said number of hematopoietic stem cells is quantified by number of CD90 positive cells in circulation.

Preferred methods include embodiments wherein said number of hematopoietic stem cells is quantified by number of interleukin-3 receptor positive cells in circulation.

Preferred methods include embodiments wherein said number of hematopoietic stem cells is quantified by number of c-kit positive cells in circulation.

Preferred methods include embodiments wherein said number of hematopoietic stem cells is quantified by number of CD34 positive, CD38 negative cells in circulation.

Preferred methods include embodiments wherein said number of hematopoietic stem cells is quantified by number of CD133 positive, CD38 negative cells in circulation.

Preferred methods include embodiments wherein said number of hematopoietic stem cells is quantified by number of CD90 positive, CD38 negative cells in circulation.

Preferred methods include embodiments wherein said number of hematopoietic stem cells is quantified by number of interleukin-3 receptor positive, CD38 negative cells in circulation.

Preferred methods include embodiments wherein said number of hematopoietic stem cells is quantified by number of c-kit positive, CD38 negative cells in circulation.

Preferred methods include embodiments wherein said number of hematopoietic stem cells is quantified by number of CD34 positive, CD14 negative cells in circulation.

Preferred methods include embodiments wherein said number of hematopoietic stem cells is quantified by number of CD133 positive, CD14 negative cells in circulation.

Preferred methods include embodiments wherein said number of hematopoietic stem cells is quantified by number of CD90 positive, CD14 negative cells in circulation.

Preferred methods include embodiments wherein said number of hematopoietic stem cells is quantified by number of interleukin-3 positive, CD14 negative cells in circulation.

Preferred methods include embodiments wherein said number of hematopoietic stem cells is quantified by number of c-kit positive, CD14 negative cells in circulation.

Preferred methods include embodiments wherein subjects identified to possess a reduced stem cell number and/or activity are treated before the space flight in order to restore and/or optimize stem cell number and/or activity.

Preferred methods include embodiments wherein an artificial intelligence system is utilized to quantify an optimum intervention for augmenting said stem cell number and/or activity, wherein said artificial intelligence system incorporates medical history of the subject, biochemical composition of the subject, oxidative stress of the subject, and inflammatory status of the subject.

Preferred methods include embodiments wherein said medical history of said subject focuses on aspects that may affect the process of hematopoiesis.

Preferred methods include embodiments wherein said aspect of said subject medical history is selected from a group comprising of: a) liver disease; b) cardiac disease; c) atherosclerosis; d) neoplasia; e) renal disease; f) muscular disorder; g) mitochondrial dysfunction; h) pulmonary dysfunction; i) neurological dysfunction; and j) autoimmune disorders.

Preferred methods include embodiments wherein said subject is assessed for circulating factors that may be suppressing hematopoietic stem cell number and/or activity.

Preferred methods include embodiments wherein said circulating factor capable of suppressing hematopoiesis is an agent that increases oxidative stress on hematopoietic stem cells.

Preferred methods include embodiments wherein said circulating factor capable of suppressing hematopoiesis is an agent that increases telomere erosion on hematopoietic stem cells.

Preferred methods include embodiments wherein said circulating factor capable of suppressing hematopoiesis is an agent that increases apoptosis of hematopoietic stem cells.

Preferred methods include embodiments wherein said circulating factor capable of suppressing hematopoiesis is an agent that increases necroptosis of hematopoietic stem cells.

Preferred methods include embodiments wherein said circulating factor capable of suppressing hematopoiesis is an agent that increases pyroptosis of hematopoietic stem cells.

Preferred methods include embodiments wherein said circulating factor capable of suppressing hematopoiesis is an agent that increases ferroptosis of hematopoietic stem cells.

Preferred methods include embodiments wherein said factor capable of suppressing hematopoiesis is TRAIL.

Preferred methods include embodiments wherein said factor capable of suppressing hematopoiesis is C reactive protein.

Preferred methods include embodiments wherein said factor capable of suppressing hematopoiesis is pentraxin.

Preferred methods include embodiments wherein said factor capable of suppressing hematopoiesis is TNF-alpha.

Preferred methods include embodiments wherein said factor capable of suppressing hematopoiesis is lymphotoxin.

Preferred methods include embodiments wherein said factor capable of suppressing hematopoiesis is TRAIL.

Preferred methods include embodiments wherein said factor capable of suppressing hematopoiesis is interleukin-1 beta.

Preferred methods include embodiments wherein said factor capable of suppressing hematopoiesis is interleukin-6.

Preferred methods include embodiments wherein said factor capable of suppressing hematopoiesis is interleukin-12.

Preferred methods include embodiments wherein said factor capable of suppressing hematopoiesis is interleukin-8.

Preferred methods include embodiments wherein said factor capable of suppressing hematopoiesis is interleukin-15.

Preferred methods include embodiments wherein said factor capable of suppressing hematopoiesis is interleukin-18.

Preferred methods include embodiments wherein said factor capable of suppressing hematopoiesis is interleukin-21.

Preferred methods include embodiments wherein said factor capable of suppressing hematopoiesis is interleukin-23.

Preferred methods include embodiments wherein said factor capable of suppressing hematopoiesis is interleukin-27.

Preferred methods include embodiments wherein said factor capable of suppressing hematopoiesis is interleukin-33.

Preferred methods include embodiments wherein said factor capable of suppressing hematopoiesis is interleukin-HMGB1.

Preferred methods include embodiments wherein said factor capable of suppressing hematopoiesis is MCP-1.

Preferred methods include embodiments wherein said factor capable of suppressing hematopoiesis is MIP-1 alpha.

Preferred methods include embodiments wherein said factor capable of suppressing hematopoiesis is MIP-1 beta.

Preferred methods include embodiments wherein said factor capable of suppressing hematopoiesis is MIP-1 delta.

Preferred methods include embodiments wherein said factor capable of suppressing hematopoiesis is prostaglandin E2.

Preferred methods include embodiments wherein said factor capable of suppressing hematopoiesis is NGF-1.

Preferred methods include embodiments wherein said artificial intelligence selects agents capable of stimulating hematopoietic stem cell numbers and/or activity.

Preferred methods include embodiments wherein said agent capable of stimulating hematopoiesis and/or hematopoietic stem cell number and/or activity is G-CSF.

Preferred methods include embodiments wherein said agent capable of stimulating hematopoiesis and/or hematopoietic stem cell number and/or activity is GM-CSF.

Preferred methods include embodiments wherein said agent capable of stimulating hematopoiesis and/or hematopoietic stem cell number and/or activity is M-CSF.

Preferred methods include embodiments wherein said agent capable of stimulating hematopoiesis and/or hematopoietic stem cell number and/or activity is flt-3 ligand.

Preferred methods include embodiments wherein said agent capable of stimulating hematopoiesis and/or hematopoietic stem cell number and/or activity is interleukin-3.

Preferred methods include embodiments wherein said agent capable of stimulating hematopoiesis and/or hematopoietic stem cell number and/or activity is prostaglandin E1.

Preferred methods include embodiments wherein said agent capable of stimulating hematopoiesis and/or hematopoietic stem cell number and/or activity is interleukin-5.

Preferred methods include embodiments wherein said agent capable of stimulating hematopoiesis and/or hematopoietic stem cell number and/or activity is interleukin-35.

Preferred methods include embodiments wherein said agent capable of stimulating hematopoiesis and/or hematopoietic stem cell number and/or activity is interleukin-9.

Preferred methods include embodiments wherein said agent capable of stimulating hematopoiesis and/or hematopoietic stem cell number and/or activity is progesterone induced blocking factor.

Preferred methods include embodiments wherein said agent capable of stimulating hematopoiesis and/or hematopoietic stem cell number and/or activity is luteinizing hormone.

Preferred methods include embodiments wherein said agent capable of stimulating hematopoiesis and/or hematopoietic stem cell number and/or activity is all trans retinoic acid.

Preferred methods include embodiments wherein said agent capable of stimulating hematopoiesis and/or hematopoietic stem cell number and/or activity is thrombopoietin.

Preferred methods include embodiments wherein said agent capable of stimulating hematopoiesis and/or hematopoietic stem cell number and/or activity is erythropoietin.

Preferred methods include embodiments wherein said agent capable of stimulating hematopoiesis and/or hematopoietic stem cell number and/or activity is monocyte conditioned media.

Preferred methods include embodiments wherein said monocyte conditioned media is generated by culture of monocytes with a toll like receptor agonist.

Preferred methods include embodiments wherein said toll like receptor agonist is a stimulator of TLR2.

Preferred methods include embodiments wherein said TLR2 stimulator is peptidoglycan.

Preferred methods include embodiments wherein said TLR2 stimulator is beta glucan.

Preferred methods include embodiments wherein said toll like receptor agonist is a stimulator of TLR3.

Preferred methods include embodiments wherein said TLR3 stimulator is poly IC.

Preferred methods include embodiments wherein said TLR3 stimulator is poly LCIC.

Preferred methods include embodiments wherein said TLR3 stimulator is double stranded RNA.

Preferred methods include embodiments wherein said TLR3 stimulator is an inactivated RNA virus.

Preferred methods include embodiments wherein said TLR3 stimulator is RGC100.

Preferred methods include embodiments wherein said TLR3 stimulator is ARNAX.

Preferred methods include embodiments wherein said TLR3 stimulator is Poly AU.

Preferred methods include embodiments wherein said toll like receptor agonist is a stimulator of TLR4.

Preferred methods include embodiments wherein said TLR4 stimulator is lipopolysaccharide.

Preferred methods include embodiments wherein said TLR4 stimulator is CRX-527.

Preferred methods include embodiments wherein said TLR4 stimulator is HMGB1.

Preferred methods include embodiments wherein said TLR4 stimulator is MPLA.

Preferred methods include embodiments wherein said TLR4 stimulator is OM-174.

Preferred methods include embodiments wherein said TLR4 stimulator is OM-294.

Preferred methods include embodiments wherein said toll like receptor agonist is a stimulator of TLR5.

Preferred methods include embodiments wherein said stimulator of TLR5 is flagellin.

Preferred methods include embodiments wherein said toll like receptor agonist is a stimulator of TLR7.

Preferred methods include embodiments wherein said stimulator of TLR7 is imiquimod.

Preferred methods include embodiments wherein said toll like receptor agonist is a stimulator of TLR9.

Preferred methods include embodiments wherein said stimulator of TLR9 is ODN2006.

Preferred methods include embodiments wherein said stimulator of TLR9 is ODN2216.

Preferred methods include embodiments wherein said stimulator of TLR9 is ODN2395.

Preferred methods include embodiments wherein said stimulator of TLR9 is CpG DNA.

Preferred methods include embodiments wherein said stimulator of TLR9 is IMO 2055.

Preferred methods include embodiments wherein said stimulator of TLR9 is EMD1201081.

Preferred methods include embodiments wherein said monocyte conditioned media is generated by culture of monocytes under hypoxic conditions while concurrently stimulating one of more TLRs.

Preferred methods include embodiments wherein said monocyte conditioned media is generated by culture of monocytes under hypoxic conditions while concurrently being exposed to IVIG.

Preferred methods include embodiments wherein IVIG is added to said monocytes under hypoxic conditions at a concentration and time point to simulate said monocytes to produce at least 100 ng of interleukin-10 per 1 million monocytes.

Preferred methods include embodiments wherein IVIG is added to said monocytes under hypoxic conditions at a concentration and time point to simulate said monocytes to produce at least 100 ng of interleukin-4 per 1 million monocytes.

Preferred methods include embodiments wherein IVIG is added to said monocytes under hypoxic conditions at a concentration and time point to simulate said monocytes to produce at least 10 ng of G-CSF per 1 million monocytes.

Preferred methods include embodiments wherein IVIG is added to said monocytes under hypoxic conditions at a concentration and time point to simulate said monocytes to produce at least 10 ng of GM-CSF per 1 million monocytes.

Preferred methods include embodiments wherein IVIG is added to said monocytes under hypoxic conditions at a concentration and time point to simulate said monocytes to produce at least 10 ng of M-CSF per 1 million monocytes.

Preferred methods include embodiments wherein IVIG is added to said monocytes under hypoxic conditions at a concentration and time point to simulate said monocytes to produce at least 20 ng of HGF per 1 million monocytes.

Preferred methods include embodiments wherein IVIG is added to said monocytes under hypoxic conditions at a concentration and time point to simulate said monocytes to produce at least 10 ng of IGF-1 per 1 million monocytes.

Preferred methods include embodiments wherein IVIG is added to said monocytes under hypoxic conditions at a concentration and time point to simulate said monocytes to produce at least 10 ng of leukemia inhibitory factor per 1 million monocytes.

Preferred methods include embodiments wherein IVIG is added to said monocytes under hypoxic conditions at a concentration and time point to simulate said monocytes to produce at least 5 ng of vascular endothelial growth factor per 1 million monocytes.

Preferred methods include embodiments wherein IVIG is added to said monocytes under hypoxic conditions at a concentration and time point to simulate said monocytes to produce at least 5 ng of epidermal growth factor per 1 million monocytes.

Preferred methods include embodiments wherein IVIG is added to said monocytes under hypoxic conditions at a concentration and time point to simulate said monocytes to produce at least 15 ng of soluble HLA-G per 1 million monocytes.

Preferred methods include embodiments wherein IVIG is added to said monocytes under hypoxic conditions at a concentration and time point to simulate said monocytes to produce at least 20 ng of soluble TNF-alpha receptor p55 per 1 million monocytes.

Preferred methods include embodiments wherein IVIG is added to said monocytes under hypoxic conditions at a concentration and time point to simulate said monocytes to produce at least 20 ng of soluble TNF-alpha receptor p75 per 1 million monocytes.

Preferred methods include embodiments wherein IVIG is added to said monocytes under hypoxic conditions at a concentration and time point to simulate said monocytes to produce at least 100 ng of interleukin-1 receptor antagonist per 1 million monocytes.

Preferred methods include embodiments wherein IVIG is added to said monocytes under hypoxic conditions at a concentration and time point to simulate said monocytes to produce at least 50 ng of endoglin per 1 million monocytes.

Preferred methods include embodiments wherein IVIG is added to said monocytes under hypoxic conditions at a concentration and time point to simulate said monocytes to produce at least 50 ng of stem cell factor per 1 million monocytes.

Preferred methods include embodiments wherein IVIG is added to said monocytes under hypoxic conditions at a concentration and time point to simulate said monocytes to produce at least 100 ng of GDF-11 per 1 million monocytes.

Preferred methods include embodiments wherein said monocyte conditioned media is generated from monocytes expressing CD14.

Preferred methods include embodiments wherein said monocyte conditioned media is generated from monocytes expressing CD16.

Preferred methods include embodiments wherein said monocyte conditioned media is generated from monocytes expressing CD90.

Preferred methods include embodiments wherein said monocyte conditioned media is generated from monocytes expressing arginase.

Preferred methods include embodiments wherein said monocyte conditioned media is generated from monocytes differentiated from bone marrow.

Preferred methods include embodiments wherein said monocyte conditioned media is generated from monocytes differentiated from peripheral blood progenitors.

Preferred methods include embodiments wherein said peripheral blood progenitors are collected after mobilization.

Preferred methods include embodiments wherein said mobilization is performed by administration of an agent that interferes with the CXCR4-SDF1 axis in the bone marrow.

Preferred methods include embodiments wherein said agent that interferes with said CXCR4-SDF1 axis is mozabil.

Preferred methods include embodiments wherein said agent that interferes with said CXCR4-SDF1 axis is ant antibody to SDF-1.

Preferred methods include embodiments wherein said agent that interferes with said CXCR4-SDF1 axis is an aptamer specific to SDF-1.

Preferred methods include embodiments wherein said agent that interferes with said CXCR4-SDF1 axis is a ribozyme specific to SDF-1.

Preferred methods include embodiments wherein said agent that interferes with said CXCR4-SDF1 axis is an antisense oligonucleotide specific to SDF-1.

Preferred methods include embodiments wherein said agent that interferes with said CXCR4-SDF1 axis is a siRNA specific to SDF-1.

Preferred methods include embodiments wherein said agent that interferes with said CXCR4-SDF1 axis is a shRNA specific to SDF-1.

Preferred methods include embodiments wherein said agent that interferes with said CXCR4-SDF1 axis is an antibody specific to CXCR4.

Preferred methods include embodiments wherein said agent that interferes with said CXCR4-SDF1 axis is an aptamer specific to CXCR4.

Preferred methods include embodiments wherein said agent that interferes with said CXCR4-SDF1 axis is a ribozyme specific to CXCR4.

Preferred methods include embodiments wherein said agent that interferes with said CXCR4-SDF1 axis is a siRNA specific to CXCR4.

Preferred methods include embodiments wherein said agent that interferes with said CXCR4-SDF1 axis is a siRNA specific to CXCR4.

Preferred methods include embodiments wherein said agent that interferes with said CXCR4-SDF1 axis is a shRNA specific to CXCR4.

Preferred methods include embodiments wherein said agent that interferes with said CXCR4-SDF1 axis is a mRNA specific to CXCR4.

Preferred methods include embodiments wherein said agent that interferes with said CXCR4-SDF1 axis is a antisense oligonucleotide specific to CXCR4.

Preferred methods include embodiments wherein said mobilization is induced by treatment of a subject with lipopolysaccharide.

Preferred methods include embodiments wherein said mobilization is induced by treatment of a subject with Poly IC.

Preferred methods include embodiments wherein said mobilization is induced by treatment of a subject with Poly IU.

Preferred methods include embodiments wherein said mobilization is induced by treatment of a subject with neupogen.

Preferred methods include embodiments wherein said mobilization is induced by treatment of a subject with leukine.

Preferred methods include embodiments wherein said mobilization is induced by treatment of a subject with monocyte colony stimulating factor.

Preferred methods include embodiments wherein said mobilization is induced by treatment of a subject with flt3 ligand.

Preferred methods include embodiments wherein said monocyte is generated from a CD34 progenitor cell.

Preferred methods include embodiments wherein said monocyte is generated from a CD133 progenitor cell.

Preferred methods include embodiments wherein said monocyte is generated from a c-kit progenitor cell.

Preferred methods include embodiments wherein said monocyte is generated from a CD127 progenitor cell.

Preferred methods include embodiments wherein said monocyte is extracted from plastic adherent peripheral blood mononuclear cells.

Preferred methods include embodiments wherein said monocyte is extracted from plastic adherent peripheral umbilical cord blood mononuclear cells.

Preferred methods include embodiments wherein said monocyte is extracted from plastic adherent peripheral menstrual blood mononuclear cells.

Preferred methods include embodiments wherein said monocyte is extracted from pluripotent stem cell derived myeloid progenitors.

Preferred methods include embodiments wherein said pluripotent stem cell is an embryonic stem cell.

Preferred methods include embodiments wherein said pluripotent stem cell is a somatic cell nuclear transfer derived stem cell.

Preferred methods include embodiments wherein said pluripotent stem cell is a parthenogenesis derived stem cell.

Preferred methods include embodiments wherein said pluripotent stem cell is an inducible pluripotent derived stem cell.

Preferred methods include embodiments wherein said inducible pluripotent stem cell is generated by retrodifferentiation of somatic cells.

Preferred methods include embodiments wherein said retrodifferentiation is accomplished by transfection with pluripotency associated genes.

Preferred methods include embodiments wherein said retrodifferentiation is accomplished by transfection with pluripotency associated microRNAs.

Preferred methods include embodiments wherein said retrodifferentiation is accomplished by transfection with pluripotency associated mRNA.

Preferred methods include embodiments wherein said retrodifferentiation is accomplished by transfection with pluripotency associated proteins.

Preferred methods include embodiments wherein said pluripotency associated gene is OCT-4.

Preferred methods include embodiments wherein said pluripotency associated gene is KLF.

Preferred methods include embodiments wherein said pluripotency associated gene is Nanog.

Preferred methods include embodiments wherein said pluripotency associated gene is SOX-2.

Preferred methods include embodiments wherein said pluripotency associated gene is k-ras.

Preferred methods include embodiments wherein said pluripotency associated gene is SSEA1.

Preferred methods include embodiments wherein said pluripotency associated gene is TRA-1.

Preferred methods include embodiments wherein said pluripotency associated gene is SSEA2.

Preferred methods include embodiments wherein said pluripotency associated gene is GDF-11 receptor.

Preferred methods include embodiments wherein said pluripotent stem cell is generated by treatment of a somatic cell with one or more pluripotency factors in the presence of a histone deacetylase inhibitor.

Preferred methods include embodiments wherein said histone deacetylase inhibitor is valproic acid.

Preferred methods include embodiments wherein said valproic acid is added to said somatic cells prior to administration of pluripotency genes.

Preferred methods include embodiments wherein said valproic acid is administered to said somatic cells at a concentration and time span sufficient to induce production of at least 10 ng/1 million cells of IL-3.

Preferred methods include embodiments wherein said valproic acid is administered to said somatic cells at a concentration and time span sufficient to induce production of at least 30 ng/1 million cells of IL-6.

Preferred methods include embodiments wherein said valproic acid is administered to said somatic cells at a concentration and time span sufficient to induce production of at least 10 ng/1 million cells of IL-10.

Preferred methods include embodiments wherein said valproic acid is administered to said somatic cells at a concentration and time span sufficient to induce production of at least 50 ng/1 million cells of soluble wnt5.

Preferred methods include embodiments wherein said valproic acid is administered to said somatic cells at a concentration and time span sufficient to induce production of at least 20 ng/1 million cells of soluble HLA-G.

Preferred methods include embodiments wherein said valproic acid is administered to said somatic cells at a concentration and time span sufficient to induce production of at least 30 ng/1 million cells of soluble trem-1.

Preferred methods include embodiments wherein said valproic acid is administered to said somatic cells at a concentration and time span sufficient to induce production of at least 10 ng/1 million cells of soluble ILT3.

Preferred methods include embodiments wherein said valproic acid is administered to said somatic cells at a concentration and time span sufficient to induce production of at least 10 ng/1 million cells of IL-35.

Preferred methods include embodiments wherein said histone deacetylase inhibitor is sodium phenylbutyrate.

Preferred methods include embodiments wherein said sodium phenylbutyrate is administered to said cells in the presence of a GSK-3 inhibitor.

Preferred methods include embodiments wherein said GSK-3 inhibitor is lithium.

Preferred methods include embodiments wherein said lithium is added to said cells together with an activator of TLR4.

Preferred methods include embodiments wherein said TLR4 activator is one or more fragments of lipopolysaccharide.

Preferred methods include embodiments wherein said TLR4 activator is low molecular weight hyaluronic acid.

Preferred methods include embodiments wherein said TLR4 activator is low molecular weight collagen fragments.

Preferred methods include embodiments wherein said TLR4 activator is low molecular weight heparin.

Preferred methods include embodiments wherein said TLR4 activator is low molecular weight syndecan.

Preferred methods include embodiments wherein said TLR4 activator is HMBG1.

Preferred methods include embodiments wherein said TLR4 activator is naltrexone.

Preferred methods include embodiments wherein said TLR4 activator is anti-TLR4 crosslinking antibody.

Preferred methods include embodiments wherein said monocyte conditioned media is generated by culture of monocytes with allogeneic T cells.

Preferred methods include embodiments wherein said T cells are administered to said monocyte cultures in the form of peripheral blood mononuclear cells.

Preferred methods include embodiments wherein said culture of monocytes and peripheral blood mononuclear cells are performed under hypoxia.

Preferred methods include embodiments wherein said culture of monocytes and peripheral blood mononuclear cells are performed under hypoxia in the presence of mesenchymal stem cells.

Preferred methods include embodiments wherein said mesenchymal stem cells are derived from bone marrow.

Preferred methods include embodiments wherein said mesenchymal stem cells are derived from umbilical cord blood.

Preferred methods include embodiments wherein said mesenchymal stem cells are derived from umbilical cord tissue.

Preferred methods include embodiments wherein said mesenchymal stem cells are derived from Wharton's Jelly.

Preferred methods include embodiments wherein said mesenchymal stem cells are derived from fallopian tubes.

Preferred methods include embodiments wherein said mesenchymal stem cells are derived from mobilized peripheral blood.

Preferred methods include embodiments wherein said mesenchymal stem cells are derived omentum.

Preferred methods include embodiments wherein said mesenchymal stem cells are derived from adipose tissue.

Preferred methods include embodiments wherein said adipose tissue is white adipose tissue.

Preferred methods include embodiments wherein said mesenchymal stem cells are derived from brown adipose tissue.

Preferred methods include embodiments wherein said mesenchymal stem cells are derived from pink adipose tissue.

Preferred methods include embodiments wherein said mesenchymal stem cells are derived from deciduous tooth.

Preferred methods include embodiments wherein said mesenchymal stem cells are derived from inducible pluripotent stem cells.

Preferred methods include embodiments wherein said mesenchymal stem cells are derived from parthenogenic stem cells.

Preferred methods include embodiments wherein said mesenchymal stem cells are derived from somatic cell nuclear transfer stem cells.

Preferred methods include embodiments wherein said mesenchymal stem cells are derived from embryonic stem cells

Preferred methods include embodiments wherein said mesenchymal stem cells are derived from very small embryonic like cells.

Preferred methods include embodiments wherein said mesenchymal stem cells express CD90.

Preferred methods include embodiments wherein said mesenchymal stem cells express c-kit.

Preferred methods include embodiments wherein said mesenchymal stem cells express CD44.

Preferred methods include embodiments wherein said mesenchymal stem cells express CD105.

Preferred methods include embodiments wherein said mesenchymal stem cells express PD-L1.

Preferred methods include embodiments wherein said mesenchymal stem cells express PD-L2.

Preferred methods include embodiments wherein said mesenchymal stem cells express FoxP3.

Preferred methods include embodiments wherein said mesenchymal stem cells express AIRE.

Preferred methods include embodiments wherein said mesenchymal stem cells express Fas ligand.

Preferred methods include embodiments wherein said mesenchymal stem cells express CD37.

Preferred methods include embodiments wherein said mesenchymal stem cells express decay accelerating factor.

Preferred methods include embodiments wherein said mesenchymal stem cells express CD73.

Preferred methods include embodiments wherein said mesenchymal stem cells express CD56.

Preferred methods include embodiments wherein said mesenchymal stem cells express CD57.

Preferred methods include embodiments wherein said mesenchymal stem cells express TREM1.

Preferred methods include embodiments wherein said mesenchymal stem cells express TIM-3.

Preferred methods include embodiments wherein said mesenchymal stem cells secrete more than 10 ng of interleukin-10 per million cells upon stimulation with 1 ng lipopolysaccharide/million cells for 24 hours.

Preferred methods include embodiments wherein said mesenchymal stem cells secrete more than 10 ng of interleukin-1 receptor antagonist per million cells upon stimulation with 1 ng lipopolysaccharide/million cells for 24 hours.

Preferred methods include embodiments wherein said mesenchymal stem cells secrete more than 10 ng of TNF-beta per million cells upon stimulation with 1 ng lipopolysaccharide/million cells for 24 hours.

Preferred methods include embodiments wherein said mesenchymal stem cells secrete more than 10 ng of VEGF per million cells upon stimulation with 1 ng lipopolysaccharide/million cells for 24 hours.

Preferred methods include embodiments wherein said mesenchymal stem cells secrete more than 10 ng of EGF per million cells upon stimulation with 1 ng lipopolysaccharide/million cells for 24 hours.

Preferred methods include embodiments wherein said mesenchymal stem cells secrete more than 10 ng of IGF-1 per million cells upon stimulation with 1 ng lipopolysaccharide/million cells for 24 hours.

Preferred methods include embodiments wherein said mesenchymal stem cells secrete more than 10 ng of interleukin-13 per million cells upon stimulation with 1 ng lipopolysaccharide/million cells for 24 hours.

Preferred methods include embodiments wherein said mesenchymal stem cells secrete more than 10 ng of angiopoietin per million cells upon stimulation with 1 ng lipopolysaccharide/million cells for 24 hours.

Preferred methods include embodiments wherein said mesenchymal stem cells secrete more than 10 ng of hepatocyte growth factor per million cells upon stimulation with 1 ng lipopolysaccharide/million cells for 24 hours.

Preferred methods include embodiments wherein said mesenchymal stem cells secrete more than 10 ng of acidic fibroblast growth factor per million cells upon stimulation with 1 ng lipopolysaccharide/million cells for 24 hours.

Preferred methods include embodiments wherein said mesenchymal stem cells secrete more than 10 ng of basic fibroblast growth factor per million cells upon stimulation with 1 ng lipopolysaccharide/million cells for 24 hours.

Preferred methods include embodiments wherein said mesenchymal stem cells secrete more than 10 ng of interleukin-20 per million cells upon stimulation with 1 ng lipopolysaccharide/million cells for 24 hours.

Preferred methods include embodiments wherein said mesenchymal stem cells secrete more than 10 ng of interleukin-22 per million cells upon stimulation with 1 ng lipopolysaccharide/million cells for 24 hours.

Preferred methods include embodiments wherein said mesenchymal stem cells secrete more than 10 ng of interleukin-35 per million cells upon stimulation with 1 ng lipopolysaccharide/million cells for 24 hours.

Preferred methods include embodiments wherein said mesenchymal stem cells secrete more than 10 ng of interleukin-38 per million cells upon stimulation with 1 ng lipopolysaccharide/million cells for 24 hours.

Preferred methods include embodiments wherein said monocytes are cultured in a media generated by an artificial intelligence system whose input is based on patient specific parameters integrating stem cell stimulatory and stem cell inhibitory host characteristics.

Preferred methods include embodiments wherein said monocytes are cultured with T cells in a manner to induce production of at least 20 ng/million cells of hepatocyte growth factor.

Preferred methods include embodiments wherein said T cells are allogeneic to said monocytes.

Preferred methods include embodiments wherein said T cells are autologous to said monocytes.

Preferred methods include embodiments wherein said T cells are xenogeneic to said monocytes.

Preferred methods include embodiments wherein said autologous T cells are activated prior to culture with said monocytes.

Preferred methods include embodiments wherein said T cells are activated by culture with allogeneic antigen presenting cells.

Preferred methods include embodiments wherein said allogeneic antigen presenting cells express at least one or more costimulatory molecules.

Preferred methods include embodiments wherein said costimulatory molecule is interleukin-1.

Preferred methods include embodiments wherein said costimulatory molecule is interleukin-2.

Preferred methods include embodiments wherein said costimulatory molecule is interleukin-12.

Preferred methods include embodiments wherein said costimulatory molecule is CD40.

Preferred methods include embodiments wherein said costimulatory molecule is CD80.

Preferred methods include embodiments wherein said costimulatory molecule is CD86.

Preferred methods include embodiments wherein said costimulatory molecule is ICOS.

Preferred methods include embodiments wherein said costimulatory molecule is ICAM-1.

Preferred methods include embodiments wherein said costimulatory molecule is LFA-1.

Preferred methods include embodiments wherein said costimulatory molecule is EP-CAM.

Preferred methods include embodiments wherein said costimulatory molecule is CD5.

Preferred methods include embodiments wherein said costimulatory molecule is CD10.

Preferred methods include embodiments wherein said costimulatory molecule is LFA-3.

Preferred methods include embodiments wherein said antigen presenting cell is capable of endocytosing antigens.

Preferred methods include embodiments wherein said antigen presenting cell is capable of activating T cells in a MHC dependent manner.

Preferred methods include embodiments wherein said antigen presenting cell is a professional antigen presenting cell.

Preferred methods include embodiments wherein said antigen presenting cell is a nonprofessional antigen presenting cell.

Preferred methods include embodiments wherein said antigen presenting cell is an endothelial cell.

Preferred methods include embodiments wherein said antigen presenting cell is an endothelial cell pretreated with interferon gamma.

Preferred methods include embodiments wherein said antigen presenting cell is a macrophage.

Preferred methods include embodiments wherein said antigen presenting cell is a macrophage pretreated with interferon gamma.

Preferred methods include embodiments wherein said antigen presenting cell is a B cell.

Preferred methods include embodiments wherein said antigen presenting cell is a B cell pretreated with interferon gamma.

Preferred methods include embodiments wherein said antigen presenting cell is a dendritic cell.

Preferred methods include embodiments wherein said antigen presenting cell is a mature dendritic cell.

Preferred methods include embodiments wherein said mature dendritic cell possesses a reduced ability to endocytose antigens.

Preferred methods include embodiments wherein said mature dendritic cell possesses a reduced migration activity.

Preferred methods include embodiments wherein said mature dendritic cell possesses enhanced ability to activate naïve T cells.

Preferred methods include embodiments wherein said mature dendritic cell possesses a reduced ability to endocytose antigens.

Preferred methods include embodiments wherein said mature dendritic cells possess enhanced ability to activate naïve T cells.

Preferred methods include embodiments wherein said mature dendritic cells possess enhanced levels of CD40.

Preferred methods include embodiments wherein said mature dendritic cells possess enhanced levels of CD46.

Preferred methods include embodiments wherein said mature dendritic cells possess enhanced levels of CD80.

Preferred methods include embodiments wherein said mature dendritic cells possess enhanced levels of CD86.

Preferred methods include embodiments wherein said mature dendritic cells possess enhanced levels of TIM3.

Preferred methods include embodiments wherein said mature dendritic cells possess enhanced levels of IL-12.

Preferred methods include embodiments wherein said mature dendritic cells possess enhanced levels of IL-15.

Preferred methods include embodiments wherein said mature dendritic cells possess enhanced levels of IL-18.

Preferred methods include embodiments wherein said mature dendritic cells possess enhanced levels of IL-17.

Preferred methods include embodiments wherein said mature dendritic cells possess enhanced levels of IL-22.

Preferred methods include embodiments wherein said mature dendritic cells possess enhanced levels of IL-27.

Preferred methods include embodiments wherein said B cell expresses CD19.

Preferred methods include embodiments wherein said B cell expresses CD20.

Preferred methods include embodiments wherein said B cell expresses CD5.

Preferred methods include embodiments wherein said B cell expresses HLA-A.

Preferred methods include embodiments wherein said B cell expresses HLA-B.

Preferred methods include embodiments wherein said B cell expresses HLA-C.

Preferred methods include embodiments wherein said B cell expresses HLA-DR.

Preferred methods include embodiments wherein said B cell expresses transporter of antigenic peptides.

Preferred methods include embodiments wherein said B cell expresses immunoglobulin on the surface.

Preferred methods include embodiments wherein said B cell secretes interleukin-10.

Preferred methods include embodiments wherein said B cell secretes interleukin-6.

Preferred methods include embodiments wherein said B cell is capable of differentiating into a plasma cell.

Preferred methods include embodiments wherein said T cell activation is induced by treatment of T cells with one or more agents capable of activating the T cell receptor.

Preferred methods include embodiments wherein said T cell activation is induced by treatment of T cells with one or more agents capable of activating CD3.

Preferred methods include embodiments wherein said T cell activation is induced by treatment of T cells with one or more agents capable of activating the T cell receptor zeta chain.

Preferred methods include embodiments wherein said T cell activation is induced by treatment of T cells with one or more agents capable of activating CD28.

Preferred methods include embodiments wherein said T cell activation is induced by treatment of T cells with one or more agents capable of activating the inducible costimulatory molecule (ICOS).

Preferred methods include embodiments wherein said T cell activation is induced by treatment of T cells with a lectin.

Preferred methods include embodiments wherein said lectin is concanavalin-A.

Preferred methods include embodiments wherein said lectin is pokeweed mitogen.

Preferred methods include embodiments wherein said lectin is phytohemagglutinin.

Preferred methods include embodiments wherein said lectin is CNA.

Preferred methods include embodiments wherein said lectin is GNA.

Preferred methods include embodiments wherein said T cell is activated by contact with an allogeneic T cell.

Preferred methods include embodiments wherein said T cell is activated by exposure to anti-CD3 antibody.

Preferred methods include embodiments wherein said anti-CD3 antibody is immobilized to a solid surface.

Preferred methods include embodiments wherein said solid surface is plastic surface.

Preferred methods include embodiments wherein said solid surface is tissue culture flask.

Preferred methods include embodiments wherein said solid surface is a microbead.

Preferred methods include embodiments wherein said solid surface is an alginate microbead.

Preferred methods include embodiments wherein said solid surface is a plant virus.

Preferred methods include embodiments wherein said solid surface is a cowpea mosaic virus.

Preferred methods include embodiments wherein said solid surface is an exosome.

Preferred methods include embodiments wherein said solid surface is a microvesicle.

Preferred methods include embodiments wherein said solid surface is a nanoparticle.

Preferred methods include embodiments wherein said solid surface is a nanotube.

Preferred methods include embodiments wherein said solid surface is a silica particle.

Preferred methods include embodiments wherein said solid surface is an artificial antigen presenting cell.

Preferred methods include embodiments wherein said solid surface is an apoptotic body.

Preferred methods include embodiments wherein said T cell is activated by ligation of CD3 using a small molecule.

Preferred methods include embodiments wherein said T cell is activated by ligation of CD3 using an aptamer.

Preferred methods include embodiments wherein said T cell is activated by ligation of CD3 using a microbody.

Preferred methods include embodiments wherein said T cell is activated by ligation of CD3 using a polypeptide.

Preferred methods include embodiments wherein said T cell is activated by ligation of CD3 using a DNano particle.

Preferred methods include embodiments wherein said T cell is activated by ligation of CD28 using an antibody.

Preferred methods include embodiments wherein said T cell is activated by ligation of CD28 using a small molecule.

Preferred methods include embodiments wherein said T cell is activated by ligation of CD28 using an aptamer.

Preferred methods include embodiments wherein said T cell is activated by ligation of CD28 using a microbody.

Preferred methods include embodiments wherein said T cell is activated by ligation of CD28 using a polypeptide.

Preferred methods include embodiments wherein said T cell is activated by ligation of CD28 using a DNano particle.

Preferred methods include embodiments wherein a system for preserving optimum health of an astronaut is disclosed comprising of: a) a communications link to one or more sensors monitoring said astronaut; b) an artificial intelligence engine coupled via the communications link to the one or more sensors detecting medical parameters on the astronaut, wherein said artificial intelligence engine using word associations for machine learning; said artificial intelligence engine receiving via the communications link audio data, visual data, and text data being sent simultaneously from medical parameter relays; c) a database storing data from a plurality of other situations in which astronauts have been in, wherein said artificial intelligence engine queries said database upon receiving an incoming data; said artificial intelligence engine analyzing said incoming data and said data from said plurality of other situations astronauts have been in by recognizing key words and patterns in aggregated and correlated data; based on said analyzing, said artificial intelligence engine generating one or more of the following medical parameters said artificial intelligence engine sending said one or more messages to a human operator; and said artificial intelligence engine receiving feedback from the human operator regarding said one or more messages; and d) wherein said system makes medical decisions autonomously and/or under the guidance of human intervention.

Preferred methods include embodiments wherein said artificial intelligence system is capable of providing in vivo real time responses to the need and/or alterations of the patient health system.

### DETAILED DESCRIPTION OF THE INVENTION

The invention provides means of leveraging deep learning and/or artificial intelligence systems to guide prophylactic and/or therapeutic interventions in a subject undergoing space travel based on genetic, biological and physiological baseline parameters and alterations of these parameters in real time as the mission is underway. In some embodiments the systems are utilized to selected candidates for suitability to enter space programs, as well as monitoring and/or treatment after completion of the space program. In some further embodiments the invention is utilized in extraterrestrial domains such as hypo or hyper gravity situations.

In one embodiment of the invention, method and means of evaluating physiological changes and modification of said changes by an artificial intelligence system is provided. Aspects of the methods include obtaining dynamic functional data from the biological system; and evaluating the homoeostatic capacity of said system from the dynamic functional data. Also provided are devices configured for use in practicing the methods. Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

The term “administered” or “administering”, as used herein, refers to any method of providing a composition to an individual such that the composition has its intended effect on the individual. For example, one method of administering is by a direct mechanism such as, local tissue administration, oral ingestion, transdermal patch, topical, inhalation, suppository etc.

As used herein, “allogeneic” refers to tissues or cells from another body that in a natural setting are immunologically incompatible or capable of being immunologically incompatible, although from one or more individuals of the same species.

As used herein, the term “allotransplantation” refers to the transplantation of organs, tissues, and/or cells from a donor to a recipient, where the donor and recipient are different individuals, but of the same species. Tissue transplanted by such procedures is referred to as an allograft or allotransplant.

As used herein, the terms “allostimulatory” and “alloreactive” refer to stimulation and reaction of the immune system in response to an allologous antigens, or “alloantigen's” or cells expressing a dissimilar HLA haplotype.

As used herein, “autologous” refers to tissues or cells that are derived or transferred from the same individual's body.

As used herein, the term “autotransplantation” refers to the transplantation of organs, tissues, and/or cells from one part of the body in an individual to another part in the same individual, i.e., the donor and recipient are the same individual. Tissue transplanted by such “autologous” procedures is referred to as an autograft or autotransplant.

The term “biologically active” refers to any molecule having structural, regulatory or biochemical functions.

The term “Cell culture” as used herein refers to an artificial in vitro system containing viable cells, whether quiescent, senescent or (actively) dividing. In a cell culture, cells are grown and maintained at an appropriate temperature, typically a temperature of 37° C. and under an atmosphere typically containing oxygen and CO2, although in other cases these are altered. Culture conditions may vary widely for each cell type though, and variation of conditions for a particular cell type can result in different phenotypes being expressed. The most commonly varied factor in culture systems is the growth medium. Growth media can vary in concentration of nutrients, growth factors, and the presence of other components. The growth factors used to supplement media are often derived from animal blood, such as calf serum.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The term “drug”, “agent” or “compound” as used herein, refers to any pharmacologically active substance capable of being administered that achieves a desired effect. Drugs or compounds can be synthetic or naturally occurring, non-peptide, proteins or peptides, oligonucleotides, or nucleotides (DNA and/or RNA), polysaccharides or sugars.

The term “individual”, as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, “mesenchymal stromal cell” or more mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

In one embodiment of the invention, the astronaut is assessed for predisposition to autoimmunity. As the astronaut is exposed to conditions that may amplify autoimmunity, the artificial intelligence provides solutions and possible prophylactic approach to the autoimmunity. One example of how space travel may alter and/or stimulate autoimmunity is through induction of lymphopenia which is known to cause homeostatic proliferation of T cells, which is a cause of autoimmunity. In one embodiments genetic abnormalities associated with autoimmunity are screening. Examples of autoimmune conditions including the following: achlorhydra autoimmune active chronic hepatitis, acute disseminated encephalomyelitis, acute hemorrhagic leukoencephalitis, Addison's disease, agammaglobulinemia, alopecia areata, Alzheimer's disease, amyotrophic lateral sclerosis, ankylosing spondylitis, anti-gbm/tbm nephritis, antiphospholipid syndrome, antisynthetase syndrome, aplastic anemia, arthritis, atopic allergy, atopic dermatitis, autoimmune cardiomyopathy, autoimmune hemolytic anemia, autoimmune hepatitis, autoimmune inner ear disease, autoimmune lymphoproliferative syndrome, autoimmune peripheral neuropathy, autoimmune polyendocrine syndrome, autoimmune progesterone dermatitis, autoimmune thrombocytopenia purpura, autoimmune uveitis, balo disease/balo concentric sclerosis, Bechets syndrome, Berger's disease, Bickerstaff's encephalitis, Blau syndrome, bullous pemphigoid, Castleman's disease, Chagas disease, chronic fatigue immune dysfunction syndrome, chronic inflammatory demyelinating polyneuropathy, chronic lyme disease, chronic obstructive pulmonary disease, Churg-Strauss syndrome, cicatricial pemphigoid, coeliac disease, Cogan syndrome, cold agglutinin disease, cranial arteritis, crest syndrome, Crohns disease, Cushing's syndrome, Dego's disease, Dercum's disease, dermatitis herpetiformis, dermatomyositis, diabetes mellitus type 1, Dressler's syndrome, discoid lupus erythematosus, eczema, endometriosis, enthesitis-related arthritis, eosinophilic fasciitis, epidermolysis bullosa acquisita, essential mixed cryoglobulinemia, Evan's syndrome, fibrodysplasia ossificans progressive, fibromyalgia, fibromyositis, fibrosing aveolitis, gastritis, gastrointestinal pemphigoid, giant cell arteritis, glomerulonephritis, Goodpasture's syndrome, Graves' disease, Guillain-barré syndrome (GBS), Hashimoto's encephalitis, Hashimoto's thyroiditis, henoch-schonlein purpura, hidradenitis suppurativa, Hughes syndrome, inflammatory bowel disease (IBD), idiopathic inflammatory demyelinating diseases, idiopathic pulmonary fibrosis, idiopathic thrombocytopeniaurpura, iga nephropathy, inflammatory demyelinating polyneuopathy, interstitial cystitis, irritable bowel syndrome (IBS), Kawasaki's disease, lichen planus, Lou Gehrig's disease, lupoid hepatitis, lupus erythematosus, ménière's disease, microscopic polyangiitis, mixed connective tissue disease, morphea, multiple myeloma, multiple sclerosis, myasthenia gravis, myositis, narcolepsy, neuromyelitis optica, neuromyotonia, occular cicatricial pemphigoid, opsoclonus myoclonus syndrome, ord thyroiditis, Parkinson's disease, pars planitis, pemphigus, pemphigus vulgaris, pernicious anaemia, polymyalgia rheumatic, polymyositis, primary biliary cirrhosis, primary sclerosing cholangitis, progressive inflammatory neuropathy, psoriasis, psoriatic arthritis, raynaud phenomenon, relapsing polychondritis, Reiter's syndrome, rheumatoid arthritis, rheumatoid fever, sarcoidosis, schizophrenia, Schmidt syndrome, Schnitzler syndrome, scleritis, scleroderma, Sjögren's syndrome, spondyloarthropathy, sticky blood syndrome, still's disease, stiff person syndrome, sydenham chorea, sweet syndrome, takayasu's arteritis, temporal arteritis, transverse myelitis, ulcerative colitis, undifferentiated connective tissue disease, undifferentiated spondyloarthropathy, vasculitis, vitiligo, Wegener's granulomatosis, Wilson's syndrome, Wiskott-Aldrich syndrome, hypersensitivity reactions of the skin, atherosclerosis, ischemia-reperfusion injury, myocardial infarction, and restenosis.

Accordingly, in some embodiments, the system described obtains dynamic functional data from the subject in various situations. Data may include cardiac performance, endothelial induction of smooth muscle relaxation, nitric oxide production, renal function, hepatic function, and/or other organs. In one embodiment, data is collected to establish a baseline of physiological parameters. This may include an evaluation of the system during and/or after the performance of a task for which one or more of the assessed parameters is required. For example, in the context of spacecraft take off, the parameter may be assessed during and/or after exposure to a hyper gravity situation, assessing physiological parameters and determining how close said parameters are altered in a manner that could be potentially dangerous. In some embodiments, a task during which a parameter is assessed is a standardized task, and, for example, reference values for the parameter assessed during the standardized task may be available. In some embodiments, a task during which a parameter is assessed is a task performed during normal use and/or operation of the system, and, for example, reference values for the parameter assessed during the task may not be available. Functional parameters that are measured may vary widely and may include quantitative and/or qualitative assessments of essentially any useful and appropriate function of a system, where examples of such parameters include, but are not limited to, those described above, and the like, and combinations thereof. Dynamic functional data may be made up of information about a single type of functional parameter, or two or more different types of functional parameters. The dynamic functional data employed in methods of the invention may thus be made up of information obtained by measuring or assessing one or more functional parameters. Alternatively, the functional parameter(s) that is monitored in order to obtain dynamic functional data may be monitored intermittently during the given period of time, i.e., it may be obtained at one or more points over the given period of time, with an interval between points at which it is not obtained. In some embodiments, the interval may vary, ranging, for example, from 0.01 sec to 60 minutes or longer, such as 0.1 to 60 s, 0.1 s to 1 s, 0.01 s to 1 s, 0.01 s to 0.1 s, 1 s to 30 s, 1 s to 15 s, 1 s to 10 s, 1 s to 5 s, 1 s to 2 s, etc. The number and/or frequency of such intervals within the period of time may correspondingly vary, ranging, for example, from 1 interval to 1000 intervals or more within the period of time, such as e.g., 1 to 1000 intervals, 1 to 500 intervals, 1 to 100 intervals, 1 to 50 intervals, 1 to 10 intervals, 1 to 5 intervals, 10 to 1000 intervals, 10 to 500 intervals, 10 to 100 intervals, 10 to 50 intervals, 100 to 1000 intervals, 100 to 500 intervals, etc.

As summarized above, dynamic functional data may include a temporal change component. A temporal change component of dynamic functional data may include essentially any change in a functional parameter that occurs, or is expected to occur, as a function of time, including where such change does or does not also involve an applied stimulus and/or withdrawal of a stimulus and/or a change in the contextual environment of the system. For example, in some instances, a change in a functional parameter that occurs, or is expected to occur, as a function of time may be influenced by or due to one or more factors, such as cardiovascular, nephrotic, hepatic, neurological or other parameters.

In some embodiments, the system described provides an output which will estimate the level of confidence concerning a possible diagnosis, injury, threat, or the nature of need for further action. In one embodiment the machine learning/artificial intelligence system aggregates and correlates data from multiple biological systems. In one embodiment the data represents biochemical parameters, in other embodiments said data include hematological and/or neurological signals.

The present invention is unique, inter alia, because of its process for conducting the machine learning, the manner in which learning occurs, and its ability to enhance medical awareness during space travel and interventions for addressing abnormalities that may occur. In one embodiment the machine learning artificial intelligence analyzes data against existing medical protocols, then it assesses the situation and makes a recommendation. Human operators will determine whether the artificial intelligence successfully transmitted the right data. Human operators will instruct the artificial intelligence one of two things: (1) You were right because of X. Continue drawing this conclusion. (2) You were wrong because of Y. Do not continue drawing this conclusion. Second, the manner in which the artificial intelligence learns is unique. The system described is created in a manner capable of analyzing medical data against standards created for use by the astronaut/space traveler. According to one aspect of the present invention, an exemplary embodiment of an apparatus for real time medical assistance includes a communications link to one or more medical doctors answering points as back up, and artificial intelligence engine, and a database. The artificial intelligence engine is coupled via the communications link to the one or more medical interventions answering points, which may possess human intelligence. Said medical interventions include direct injection via an automated means of agents needed to counteract various physiological changes occurring in real time during the space flight take off, the actual space flight and the return.

The artificial intelligence engine uses word associations for machine learning. The artificial intelligence engine receives, via the body sensors and communications portals, audio data, visual data, and text data being sent simultaneously to one or more medical doctor points or nodes. The database stores data from a plurality of other medical situations associated with space travel. The artificial intelligence engine queries said database upon receiving an incoming data. The artificial intelligence engine analyses said incoming data and said data from said plurality of other medical conditions associated with space flight by recognizing key words and patterns in aggregated and correlated data. Based on said analyzing, said artificial intelligence engine generates one or more of the following messages: an audio message, a visual message, and a text message. Said artificial intelligence engine sends said one or more messages to a human doctor, and receives feedback from the human doctor regarding said one or more messages. In the above exemplary embodiment, the feedback may includes one or more of the following: (i) the message was correct because of X; continue drawing this conclusion; and (ii) the message was wrong because of Y; do not continue drawing this conclusion. In the above exemplary embodiment, the communication link may also be connected to one or more cardiac, hepatic and renal systems in addition to the one or more assessment means.

Assessment of physiological parameters of the astronaut can be performed with various technologies and utilizing various markers. In one embodiment the artificial intelligence is used to assessing timing and circumstances associated with hypoxia. Augmentation of hypoxia is associated with lactate dehydrogenase augmentation.

LDH present in the blood stream indicates hypoxia severe enough to reduce the blood flow to peripheral organs. This is the onset on leakage of LDH from these cells. By detecting LDH it is not possible to determine which organ that is suffering from hypoxia. If haemolysis has occurred (rupture of red blood cells will also cause an increase in LDH, even if it is not by hypoxia). Haemolysis is divided into two groups: (1) In vitro, which means that the haemolysis occur when taking the sample or at storage in test tubes, and (2) In vivo which means that the red blood cells within the patient have ruptured due to illness. An ongoing haemolysis will then give falsely high levels of LDH, not due to only hypoxia. The half-life (T½) for LDH is dependent on which type of the five isomers of LDH that has leaked out into the blood. LDH1 which is mainly present in the heart, brain and red blood cells has T½ 120 h while LDH5 mainly appears in the liver and muscles have T½ at 10 h.

In the area of space travel, the invention combines the utilization of artificial intelligence together with real time blood analysis. One medical condition that the invention provides a means to address is hypoxia. There are numerous conditions associated with space travel that predispose to hypoxia, these include stroke, cardiac ischemia, systemic hypoperfusion, and blood accumulation as a result of hypergravity. The detection of ischemia by the Al system disclosed in the invention may utilize various means of assessing suboptimal oxygen in a quantitative manner. This is fundamentally important because in some situations that oxygen sensing areas of the brain are not functioning, resulting in “happy hypoxia” in which patients do not feel that they are hypoxia and die suddenly. In one embodiment, hypoxia in an astronaut is quantified by LDH, and optionally also AST, ALT, Mg, and lactate, which can be analyzed within minutes (or alternatively in seconds) in 10 microliters of whole blood. These analyses can be measured together with free haemoglobin to make sure that haemolysis is not present with false increased values of LDH as the result. In one aspect, the invention comprises a method of determining hypoxia. According to certain embodiments, the method can comprise collecting a blood sample; and determining the total amount of LDH in the blood. The blood sample can be collected from any mammal or alternatively from any organ to be transplanted into a mammal in need thereof. In another embodiment, the method of determining hypoxia comprises collecting a blood sample, wherein the blood sample includes plasma and blood cells. Preferably, the plasma is separated from the blood cells such that the plasma can be analyzed without the presence of blood cells. The isolated plasma can be analyzed for the total amount of LDH in the plasma. As such, a determination of LDH can be achieved. Based on the LDH quantity, or alternatively in conjunction with other prognostic markers, a determination of hypoxia can be readily realized. In another embodiment, the determination of hypoxia includes analyzing a blood sample to determine the quantity or amount of multiple prognostic markers. In one such embodiment, the method comprises determining the amount of LDH in a blood sample, or preferably the plasma thereof, and determining the amount of at least one additional prognostic marker selected from the group consisting essentially of K, Mg, Ca, AST, ALT and lactate. Preferably, the blood sample for analysis is manipulated such that the plasma is separated from the blood cells. In one embodiment, this separation can be achieved by use of a semi-permeable membrane or centrifuge. When utilizing a semi-permeable membrane, the plasma preferably passes through the membrane and the blood cells are retained on the membrane. Accordingly, the plasma is for all practical purposes isolated from the bulk of the blood cells. The plasma can then be sampled and tested in the absence of blood cells. According to various embodiments, the sample volume for analysis can be greatly reduced from prior methods. In one embodiment, the volume of blood for determining hypoxia comprises from 5 μL to 60 μL, or from 5 μL to 25 μL, or preferably from about 5 to 15 μL, and in particular 10 μL. In certain embodiments, the volume of blood for determining hypoxia comprises from about 5 μL to 150 μL, or from 10 μL to 120 μL, or from 10 μL to 100 μL, or from 10 μL to 80 μLL.

At a high level, the AI engine may include at least one machine learning model that is trained to use causal inference to generate candidate drug compounds. One of the challenges with discovering new therapeutics may include determining whether certain ingredients are causal agents with respect to certain activity in a design space. The sheer number of possible sequences of ingredients may be extraordinarily large due to mathematical combinatorics, such that identifying a cause and effect relationship between ingredients and activity may be impossible or, at best, extremely unlikely, to identify without the disclosed embodiments. Based on advances in computing hardware (e.g., graphic processing unit processing cores) and the AI techniques using causal inference described herein, the disclosed embodiments may enable the efficient solving of the task of generating candidate drug compounds at scale.

By simulating numerous alternative scenarios to further optimize and hone the accuracy of a sequence of ingredients in the candidate drug compounds, such techniques may enable reducing the number of viable candidate drug compounds. As a result, the embodiments may provide technical benefits, such as reducing resources consumed (e.g., processing, memory, network bandwidth) by reducing a number of candidate drug compounds that may be considered for classification as a selected candidate drug compound by another machine learning model. In some embodiments, one application for the AI engine to design, discover, develop, formulate, create, and/or test candidate drug compounds may pertain to peptide therapeutics. A peptide may refer to a compound consisting of two or more amino acids linked in a chain. Example peptides may include dipeptides, tripeptides, tetrapeptides, etc. Aa polypeptide may refer to a long, continuous, and unbranched peptide chain. Peptides may be simple to manufacture at discovery scale, include drug-like characteristics of small molecules, include safety and high specificity of biologics, and/or provide greater administration flexibility than some other biologics. Compounds may be tested in various stem cell differentiation assays as well as stem cell proliferation assays. The utilization of stem cells may involve hematopoietic stem cells, which typically express markers such as the adhesion molecule CD34, or other ones such as CD133 and/or CD105. In some cases modulation of marker expression is desired by the drugs being screened. For example, in some embodiments it is desired to increase homing of stem cells to a desired location, so increased CXCR4 is desired.

The discriminator may use a gradient of an objective function to increase the value of the output. The discriminator may be trained as an unsupervised “density estimator,” i.e., a contrast function produces a low value for desired data (e.g., candidate drug compounds that include sequences producing desired levels of certain types of activity in a design space) and higher output for undesired data (e.g., candidate drug compounds that include sequences producing undesirable levels of certain types of activity in a design space). The generator may receive the gradient of the discriminator with respect to each modified candidate drug compound it produces. The generator uses the gradient to train itself to produce modified candidate drug compounds that the discriminator determines include sequences producing desired levels of certain types of activity in a design space. Recurrent neural networks include the functionality, in the context of a hidden layer, to process information sequences and store information about previous computations. As such, recurrent neural networks may have or exhibit a “memory.” Recurrent neural networks may include connections between nodes that form a directed graph along a temporal sequence. Keeping and analyzing information about previous states enables recurrent neural networks to process sequences of inputs to recognize patterns (e.g., such as sequences of ingredients and correlations with certain types of activity level). Recurrent neural networks may be similar to Markov chains. For example, Markov chains may refer to stochastic models describing sequences of possible events in which the probability of any given event depends only on the state information contained in the previous event. Thus, Markov chains also use an internal memory to store at least the state of the previous event. These models may be useful in determining causal inference, such as whether an event at a current node changes as a result of the state of a previous node changing.

In some embodiments the artificial intelligence system provides instructions on the use of stem cells or various derivatives of stem cells. In embodiments of the invention where specific cellular physical properties are the basis of differentiating between cord blood stem cells with various biological activities, discrimination on the basis of physical properties can be performed using a Fluorescent Activated Cell Sorter (FACS), through manipulation of the forward scatter and side scatter settings. Other methods of separating cells based on physical properties include the use of filters with specific size ranges, as well as density gradients and pheresis techniques. When differentiation is desired based on electrical properties of cells, techniques such as electrophotoluminescence may be used in combination with a cell sorting means such as FACS. Selection of cells based on ability to uptake certain compounds can be performed using, for example, the ALDESORT system, which provides a fluorescent-based means of purifying cells with high aldehyde dehydrogenase activity. Cells with high levels of this enzyme are known to possess higher proliferative and self-renewal activities in comparison to cells possessing lower levels. Other methods of identifying cells with high proliferative activity includes identifying cells with ability to selectively efflux certain dyes such as rhodamine-123 and or Hoechst 33342. Without being bound to theory, cells possessing this property often express the multidrug resistance transport protein ABCG2, and are known for enhanced regenerative ability compared to cells which do not possess this efflux mechanism. In other embodiments cord blood cells are purified for certain therapeutic properties based on expression of markers. In one particular embodiment, cord blood cells are purified for the phenotype of endothelial precursor cells. Said precursors, or progenitor cells express markers such as CD133, and/or CD34. Said progenitors may be purified by positive or negative selection using techniques such as magnetic activated cell sorting (MACS), affinity columns, FACS, panning, or by other means known in the art. Cord blood derived endothelial progenitor cells may be administered directly into the target tissue for ED, or may be administered systemically. Another variation of this embodiment is the use of differentiation of said endothelial precursor cells in vitro, followed by infusion into a patient. Verification for endothelial differentiation may be performed by assessing ability of cells to bind FITC-labeled Ulex europaeus agglutinin-1, ability to endocytose acetylated Di-LDL, and the expression of endothelial cell markers such as PECAM-1, VEGFR-2, or CD31. Certain desired activities can be endowed onto said cord blood stem cells prior to administration into the patient. In one specific embodiment cord blood cells may be “activated” ex vivo by a brief culture in hypoxic conditions in order to upregulate nuclear translocation of the HIF-1 transcription factor and endow said cord blood cells with enhanced angiogenic potential. Hypoxia may be achieved by culture of cells in conditions of 0.1% oxygen to 10% oxygen, preferably between 0.5% oxygen and 5% oxygen, and more preferably around 1% oxygen. Cells may be cultured for a variety of timepoints ranging from 1 hour to 72 hours, more preferably from 13 hours to 59 hours and more preferably around 48 hours. Assessment of angiogenic, and other desired activities useful for the practice of the current invention, can be performed prior to administration of said cord blood cells into the patient.

1. A method for maintaining optimum health of an astronaut during a space mission comprising the steps of: a) establishing a genetic and biological background on said astronaut; b) establishing a baseline level of physiological and biological features of said astronaut; c) monitoring said physiological and biological features in real time during space flight; d) intervening based on algorithms and/or artificial intelligence mediated instructions; and e) adjusting the interventions based on physiological and/or biological responses to said intervention.

2. The method of claim 1, wherein said genetic background is a polymorphism altering function of one or more organs.

3. The method of claim 1, wherein said genetic background comprises a reduces generation of cells with regenerative potential.

4. The method of claim 3, wherein said cells with regenerative potential are endogenous stem cells.

5. The method of claim 4, wherein said endogenous stem cells are capable of asymmetric division.

6. The method of 5, wherein said asymmetric division is associated with activation of the telomerase enzyme.

7. The method of 6, wherein said asymmetric division occurs in a manner in which said endogenous stem cell makes a copy of itself, as well as a cell with differentiated characteristics.

8. The method of claim 4, wherein said endogenous stem cells are found in areas of relative hypoxia.

9. The method of claim 4, wherein said endogenous stem cell possesses higher levels of immune suppressive molecules as compared to non-stem cell cells.

10. The method of claim 4, wherein said endogenous stem cells express CXCL16.

11. The method of claim 4, wherein said endogenous stem cells express ENA-78.

12. The method of claim 4, wherein said endogenous stem cells express TRANCE.

13. The method of claim 1, wherein subjects identified to possess a reduced stem cell number and/or activity are treated before the space flight in order to restore and/or optimize stem cell number and/or activity.

14. The method of claim 13, wherein an artificial intelligence system is utilized to quantify an optimum intervention for augmenting said stem cell number and/or activity, wherein said artificial intelligence system incorporates medical history of the subject, biochemical composition of the subject, oxidative stress of the subject, and inflammatory status of the subject.

15. The method of claim 14, wherein said medical history of said subject focuses on aspects that may affect the process of hematopoiesis.

16. The method of claim 15, wherein said aspect of said subject medical history is selected from a group comprising of: a) liver disease; b) cardiac disease; c) atherosclerosis; d) neoplasia; e) renal disease; f) muscular disorder; g) mitochondrial dysfunction; h) pulmonary dysfunction; i) neurological dysfunction; and j) autoimmune disorders.

17. The method of claim 14, wherein said artificial intelligence selects agents capable of stimulating hematopoietic stem cell numbers and/or activity.

18. The method of claim 17, wherein said agent capable of stimulating hematopoiesis and/or hematopoietic stem cell number and/or activity is interleukin-35.

19. The method of claim 1, wherein a system for preserving optimum health of an astronaut is disclosed comprising of: a) a communications link to one or more sensors monitoring said astronaut; b) an artificial intelligence engine coupled via the communications link to the one or more sensors detecting medical parameters on the astronaut, wherein said artificial intelligence engine using word associations for machine learning; said artificial intelligence engine receiving via the communications link audio data, visual data, and text data being sent simultaneously from medical parameter relays; c) a database storing data from a plurality of other situations in which astronauts have been in, wherein said artificial intelligence engine queries said database upon receiving an incoming data; said artificial intelligence engine analyzing said incoming data and said data from said plurality of other situations astronauts have been in by recognizing key words and patterns in aggregated and correlated data; based on said analyzing, said artificial intelligence engine generating one or more of the following medical parameters said artificial intelligence engine sending said one or more messages to a human operator; and said artificial intelligence engine receiving feedback from the human operator regarding said one or more messages; and d) wherein said system makes medical decisions autonomously and/or under the guidance of human intervention.

20. The method of claim 19, wherein said artificial intelligence system is capable of providing in vivo real time responses to the need and/or alterations of the patient health system.
